(19) United States
(12) Patent Application Publication
(10) Pub. No.: US 2012/0157536 A1
(43) Pub. Date: Jun. 21, 2012

(54) COMPOSITION FOR TRANSDERMAL ADMINISTRATION OF NON-STEROIDAL ANTI-FLAMMATORY DRUG

(75) Inventor: Kishore R. SHAH, Bridgewater, NJ (US)

(73) Assignee: Polytherapeutics, Inc., Bridgewater, NJ (US)

(21) Appl. No.: 13/327,180

(22) Filed: Dec. 15, 2011

Related U.S. Application Data
(60) Provisional application No. 61/459,779, filed on Dec. 20, 2010.

(51) Int. Cl. A61K 31/196; A61P 25/00; A61P 29/00

(52) U.S. Cl. 514/567

(57) ABSTRACT

This invention pertains to compositions and method for transdermal administration of non-steroidal anti-inflammatory and analgesic drugs (NSAID) for the treatment of inflammation and pain caused by conditions such as arthritis, degenerative joint disease, minor strains, pains, and contusions. This invention particularly relates to transdermal compositions comprising an NSAID, a bioadhesive graft copolymer, and a skin penetration enhancer, selected from pyrrolidone or its derivatives and dialkyl sulfoxides and combinations thereof.

### CROSS REFERENCE TO RELATED APPLICATIONS

This application claims the benefit of U.S. Provisional Patent Application No. 61/459,779, filed Dec. 20, 2010, the disclosure of which is incorporated by reference herein in its entirety.

### FIELD OF THE INVENTION

This invention pertains to compositions and method for transdermal administration of non-steroidal anti-inflammatory and analgesic drugs (NSAID) for the treatment of inflammation and pain caused by conditions such as arthritis, degenerative joint disease, minor strains, pains, and contusions. This invention particularly relates to transdermal compositions comprising an NSAID, a bioadhesive graft copolymer, and a skin penetration enhancer, selected from pyrrolidone or its derivatives and dialkyl sulfoxides and combinations thereof.

### BACKGROUND OF THE INVENTION

Inflammation and pain due to conditions such as rheumatoid arthritis and osteoarthritis, including degenerative joint disease of the hip and knees, minor strains, sprains, and contusions are common occurrences in both animals and human beings. Non-steroidal anti-inflammatory drugs (NSAIDs) are frequently administered for the treatment of such conditions. Main classes of NSAIDs include salicylates (e.g. aspirin), propionic acid derivatives (e.g. ibuprofen), aniline derivatives (e.g. aminophenolacetaminophen [TYLENOL®]), pyrazole derivatives (such as phenylbutazone), fenamates (e.g. meclofenamate), indole derivatives (e.g. indomethacin), acetic acid derivatives (e.g. diclofenac), oxicam derivatives (e.g. piroxicam), and cylooxygenase-2 (COX-2) inhibitors (e.g. celecoxib). The most common route of drug administration is oral ingestion of tablets or capsules. Oral administration of most of the NSAIDS, with the exception of COX-2 inhibitors and TYLENOL®, can cause severe side reactions such as gastrointestinal bleeding and ulceration, liver and kidney damages, and central nervous system and cutaneous disturbances, particularly after extended use. COX-2 inhibitor drugs have somewhat elevated cardiovascular risks in comparison with other NSAIDs. Therefore, their usage has greatly decreased in recent years. Transdermal delivery of an NSAID can alleviate such side reactions because they bypass the GI (gastrointestinal) passage, avoid first pass hepatic metabolism, and do not produce high peak of drug concentration in the blood circulation that are associated with orally administered drug.

Scientific and commercial interest in topical transdermal administration of NSAIDs has gained in importance because of the above-enunciated rationale. Transdermal dosage forms disclosed and/or commercially used include plaster, patch, gel, and solution of the drug formulated with pharmaceutically acceptable ingredients. These preparations also include enhancers for accelerating the rate of permeation of the drug through the skin. The majority of these compositions are intended to provide therapeutic effectiveness in body tissues in the vicinity of the topical skin site of treatment.

Topical formulations of NSAIDs for local activity, i.e. analgesic effect at the site of application, have been commercially available worldwide for some time. These products include FELDENE® Gel (Pfizer) with piroxicam, FLECTOR® Patch (Alpharma) with diclofenac, VOLTAREN® Gel (Novartis) with diclofenac, and recently marketed PENNSAID®(Nuvo Research) solution containing diclofenac with dimethyl sulfoxide (DMSO) as skin penetration enhancer.

Okuyama et al. (U.S. Pat. No. 5,208,035) have claimed a plaster comprising a paste spread on a backing material, said paste comprising diclofenac sodium, a penetration enhancer composed of 1-menthol and propylene glycol, and a hydrophilic base composed principally of a water-soluble polymer. The patent discloses that good percutaneous absorption of diclofenac is obtained by application of the plaster to the skin. Upon application of the patch on the back of guinea pig, systemic drug concentrations were found.

M. Akazawa (U.S. Pat. No. 5,607,690) has claimed an anti-inflammatory and analgesic plaster preparation comprising a salt of diclofenac and a cyclic organic base such as hydroxyethylpyrrolidine or hydroxyethylpiperidine having a pH in the range of 7.3 to 9.

Effing, et al. (U.S. Pat. No. 6,193,996) claims a pressure-sensitive adhesive comprising an alkyl acrylate copolymer, mixture of penetration enhancers comprising N-alkyl 2-pyrrolidone and an alkyl ester of monocarboxylic acid, an alkane polyol, and therapeutically effective amount of diclofenac or its salt. It is further disclosed that a drug delivery device comprised of the said adhesive formulation would be suitable for providing therapeutically effective systemic blood levels of the drug.

Sasaki, et al. (U.S. Pat. No. 7,651,700) have disclosed a pressure-sensitive adhesive patch containing diclofenac sodium, N-methyl 2-pyrrolidone or a derivative thereof, propylene glycol monocaprylate, and citric acid. The patch is stated to exhibit excellent releasability and percutaneous absorbability of diclofenac sodium through rat abdominal skin.

Liebschutz, et al. (U.S. Patent Application Publication No. 2004/0037872) have described a transdermal patch comprising an impermeable backing layer, diclofenac drug containing adhesive matrix layer, and a protective layer, which can be pulled-off prior to application of the matrix layer to the affected skin site. The adhesive layer also contains one or more solvents selected from the group consisting of oleic acid, fatty acid alkyl esters and N-alkyl-pyrrolidone.

Fankhauser (U.S. Pat. No. 4,999,379) has claimed a transdermal dioclofenac composition containing N,N-dimethyllauroylamide or 1-n-dodecylazacycloheptan-2-one as skin penetration enhancers.

Kasai, et al. (U.S. Pat. No. 5,350,769) have claimed an anti-inflammatory gel comprising a sodium or ammonium salt of diclofenac, a nonionic polymer such as hydroxyethyl cellulose, an ester of dibasic acid such as diisopropyl adipate, and a lower alcohol such as ethanol. Properties of the claimed gel were its stability and anti-inflammatory action.

Betlach (U.S. Pat. No. 5,374,661) discloses a topical drug delivery composition for transdermally delivering effective amounts of diclofenac via a gel. Diclofenac sodium is solubilized in a mixture of water, a low molecular weight alcohol, and a glycol. Sekine, et al. (U.S. Pat. No. 6,054,484) have claimed a transparent aqueous solution comprising diclofenac sodium, a fatty acid dialkylolamide, and water.

Jun, et al. (U.S. Pat. No. 6,368,618) have claimed a topical formulation for delivery of nonsteroidal anti-inflammatory drugs (NSAID). A two-phase liquid composition has aqueous and oil phases, the oil phase having a relatively high concentration of the NSAID to enhance transdermal absorption and efficacy when incorporated into the topical anti-inflammatory formulation. The two-phase liquid composition preferably contains, in addition to an NSAID, at least one melting point depressing agent. A preferred topical anti-inflammatory composition includes S(+)-ibuprofen, thymol, and ethyl alcohol or isopropyl alcohol.

Lee, et al. (U.S. Pat. No. 7,132,452) discloses a topical gel for the treatment of pain and inflammation associated with infection caused by herpes virus comprising diclofenac, L-menthol, propylene glycol, triethanolamine, carboxypolymethylene, isopropyl alcohol, and purified water.

Ikeda, et al. (U.S. Pat. No. 5,422,102) have disclosed an anti-inflammatory gel comprising diclofenac, an ester of dibasic acid, a lower alcohol, and a non-ionic polymer selected from hydroethyl cellulose and hydroxypropyl cellulose or mixtures thereof.

Kisak, et al. (U.S. Patent Application 2008/0300311) have disclosed a gel formulation, comprising: (i) diclofenac sodium; (ii) DMSO; (ii) ethanol; (iii) propylene glycol; (iv) optionally glycerol; (v) a thickening agent, wherein the thickening agent is selected from the group consisting of cellulose polymers, carbomer polymers, a carbomer derivative, a cellulose derivative, polyvinyl alcohol, poloxamers, polysaccharides, and mixtures thereof; and (vi) water.

### SUMMARY OF THE INVENTION

It has been determined that formulations comprising an NSAID, a skin permeation enhancer, and a film forming bioadhesive hydrophilic graft copolymer, which has a hydrophilic polymeric main chain and polystyrene side chains, provides significantly greater flux of the NSAID compared to the formulation without the bioadhesive graft copolymer.

The present invention pertains to transdermal compositions comprising a non-steroidal anti-inflammatory drug (NSAID), a skin penetration enhancer, and a film forming bioadhesive hydrophilic graft copolymer. Non-steroidal anti-inflammatory drugs suitable for use in the compositions of the present invention include without limitation diclofenac [2-(2,6-dichloro-anilino)-benzeneacetic acid], fluorbiprofen, piroxicam, ketoprofen, indomethacin, ibuprofen, meclofenamate, aminophenolacetaminophen [TYLENOL®], ketorolac, naproxen, celecoxib, and tolmetin. The skin penetration enhancer includes pyrrolidone and related compounds, dimethyl sulfoxide, decylmethyl sulfoxide, and mixtures thereof. Film forming bioadhesive hydrophilic graft copolymers suitable for use in the disclosed compositions have a hydrophilic polymeric main chain and a hydrophobic polystyrene graft chain (FIG. 1). The main chain is comprised of monomeric units having acidic groups and optionally neutral monomeric units.

It is an object of this invention to provide a transdermal composition for application to an animal (i.e., human) skin for the treatment of inflammation and/or pain. It is also an object of this invention to transdermally deliver the drug for therapeutic effect at the local site of application or for systemic absorption and efficacy throughout the body at sites distal to the site of application of the transdermal composition. Another object of the present transdermal compositions is to provide a sustained therapeutic effect, which at once improves the drug effectiveness, patient convenience and compliance. A further object of this invention is to decrease undesirable side effects, such as irritation of the gastrointestinal tract and liver toxicity, which are sometimes associated with oral administration of NSAID. Yet another object of the invention is to provide a method of transdermal administration of an NSAID for either local or systemic therapeutic effect by application of the transdermal composition to skin.

The transdermal composition can be formulated into any of the conventional topical pharmaceutical dosage forms, which include a solution, gel, emulsion, suspension, or an ointment. The dosage form of the transdermal composition, upon application to skin, dries to form a bioadherent polymeric film incorporating the drug, the skin permeation enhancer, and non-volatile excipients of the composition. The drug is delivered to the body from the polymeric film by a process of diffusion through the skin.

It has been found that the transdermal compositions of this invention greatly increase the rate of NSAID permeation through skin.

The transdermal compositions of this invention may be packaged in any convenient container, which includes without limitation tube, dropper bottle, unit dose packet, and metered multi-dose pump dispenser.

Further embodiments, features, and advantages of the embodiments, as well as the structure and operation of the various embodiments, are described in detail below with reference to accompanying drawings.

### BRIEF DESCRIPTION OF THE DRAWINGS

FIG. 1 shows a bioadhesive hydrophilic graft copolymer for use in exemplary compositions described herein.

FIG. 2 shows a polystyrene ethyl methacrylate macromonomer for use in exemplary compositions described herein.

FIG. 3 shows a bioadhesive graft copolymer (PHARMADUR®) for use in exemplary compositions described herein.

FIG. 4 shows a plot of cumulative amounts of diclofenac permeated through the skin versus time.

### DETAILED DESCRIPTION OF THE INVENTION AND PREFERRED EMBODIMENTS

It should be appreciated that the particular implementations shown and described herein are examples and are not intended to otherwise limit the scope of the application in any way.

The published patents, patent applications, websites, company names, and scientific literature referred to herein are hereby incorporated by reference in their entirety to the same extent as if each was specifically and individually indicated to be incorporated by reference. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter.

As used in this specification, the singular forms “a,” “an” and “the” specifically also encompass the plural forms of the terms to which they refer, unless the content clearly dictates otherwise. The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

Technical and scientific terms used herein have the meaning commonly understood by one of skill in the art to which the present application pertains, unless otherwise defined. Reference is made herein to various methodologies and materials known to those of skill in the art.

The present invention is directed to a transdermal composition of a non-steroidal anti-inflammatory drug (NSAID) for application to an animal skin (in (including mammals and humans) for the treatment of inflammation and/or pain caused by conditions such as arthritis, degenerative joint disease, minor strains, pains, and contusions. It is also an object of this invention to transdermally deliver the drug for therapeutic effect at the local site of application or for systemic absorption and efficacy throughout the body. A further object of this invention is to greatly increase the rate of NSAID permeation through skin.

This object is achieved with the transdermal compositions of the present invention, which suitably comprise the following:

(a) a non-steroidal anti-inflammatory drug,

(b) a skin permeation enhancer, and

(c) a film forming bioadhesive hydrophilic graft copolymer.

The compositions may contain other pharmaceutically acceptable excipients for formulation into any conventional pharmaceutically acceptable dermal dosage forms.

The following definitions are used herein for the purpose of describing the present invention:

“Drug” is used synonymously with active pharmaceutical ingredient (API), which can produce pharmacological effect in the body. In the present invention, a suitable API is an NSAID.

“Transdermal” is defined as transport of a substance, such as a drug, through the skin by a process of diffusion.

“Transdermal administration” means delivery of the drug to the body transdermally.

“Skin permeation” and “skin penetration” are used interchangeable throughout to mean transport of a substance through the skin.

“Skin permeation rate” of drug means the rate of diffusion of the drug through the skin. This rate may also be referred to as “flux” or a symbol “J” and is typically expressed as micrograms/square centimeter (μg/cm²) of skin surface/hour.

“Skin permeation enhancer,” “skin penetration enhancer” or simply the term “enhancer” are used interchangeably to mean a chemical substance or a mixture of one or more substances, which increase the rate of skin permeation of the drug.

“Solvent” is a pharmaceutically acceptable liquid substance (e.g. ethanol and/or isopropyl alcohol), which help to keep the drug in a dissolved state in the compositions of this invention.

“Systemic” therapeutic effect is associated with absorption of the drug into blood circulation to produce drug concentrations in the blood that result in pharmacological activity of the drug throughout the body and not only at the site of application of the transdermal composition.

“Local” therapeutic effect is pharmacological activity of the drug at the site of application of the transdermal composition. It includes drug delivery to tissues in the vicinity of the skin but with minimal systemic therapeutic effect.

“Excipient” includes all ingredients of the transdermal composition other than the drug, the skin permeation enhancer, and the bioadhesive graft copolymer. Excipients are generally regarded as inert and do not have an effect on the rate of skin permeation of the drug or its resulting therapeutic effect.

“Vehicle” means a composition comprising all the ingredients of the transdermal composition other than the drug.

Suitable non-steroidal anti-inflammatory drugs for use in the compositions of the present invention include without limitation, diclofenac [2-(2,6-dichloro-anilino)-benzeneacetic acid], fluorbiprofen, piroxicam, ketoprofen, indomethacin, ibuprofen, meclofenamate, aminophenolacetaminophen [TYLENOL®], ketorolac, naproxen, celecoxib, and tolmetin. Diclofenac in the form of its salt, namely sodium, potassium, ammonium, or diethyl ammonium is the preferred NSAID. A particularly preferred form of diclofenac is its monosodium salt. Concentration of the drug can range from 0.1% to 10%, based on the total weight of the composition. Suitably, the concentration of the drug ranges from about 0.2% to about 9%, or about 0.3% to about 8%, about 0.4% to about 7%, about 0.5% to about 6%, about 0.5% to about 5%, or about 0.5%, about 1%, about 1.5%, about 2%, about 2.5%, about 3%, about 3.5%, about 4%, about 4.5%, or about 5%, based on the total weight of the composition.

Unless otherwise stated, percentages (%) of components provided throughout the specification are indicated as weight percentages, based on the total weight of the composition.

Skin permeation enhancers for use in the transdermal compositions include without limitation, 2-pyrrolidone, N-methyl 2-pyrrolidone (NMP), N-octyl 2-pyrrolidone, N-dodecyl-2-pyrrolidone, N-(2-hydroxyethyl)-2-pyrrolidone, dimethyl sulfoxide (DMSO), decylmethyl sulfoxide, and mixtures thereof. A particularly preferred enhancer is a mixture of NMP and DMSO having a ratio of weight/weight within the range of NMP:DMSO=1:3 to 3:1, e.g., about 1:2, about 1.5:2.5, about 2:1, or about 2.5:1.5. Concentration of the enhancer suitably ranges from about 2% to 50%, based on the total weight of the composition. Suitably, the concentration of the enhancer ranges from about 4% to about 40%, about 5% to about 40%, or about 5%, about 10%, about 15%, about 20%, about 25%, about 30%, about 35%, or about 40%, based on the total weight of the composition.

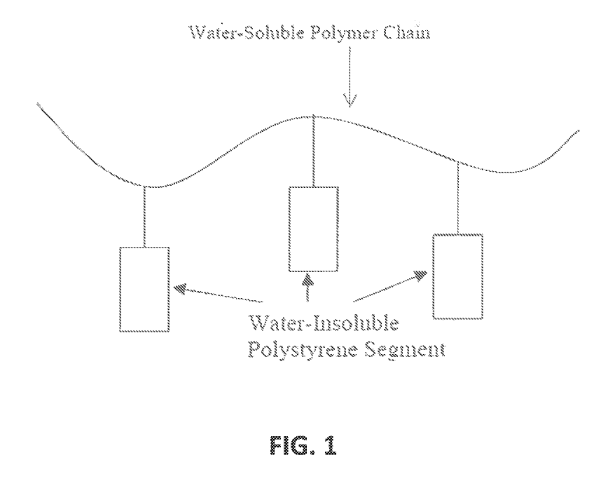

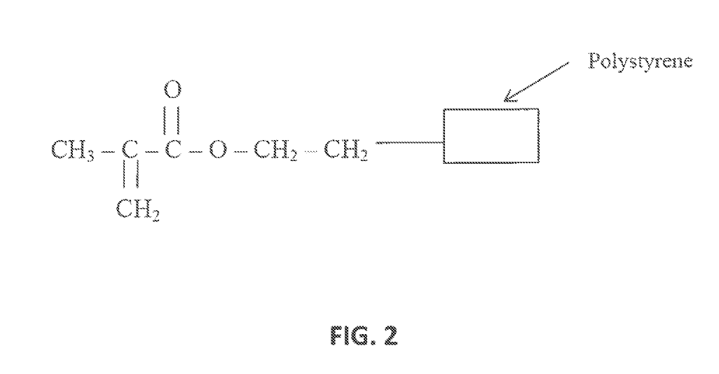

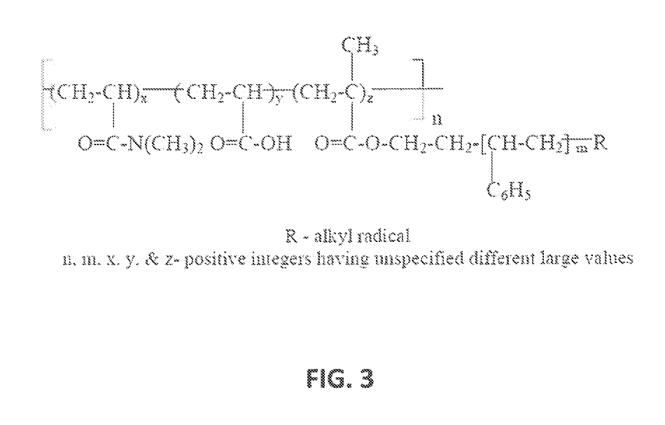

A film forming bioadhesive hydrophilic graft copolymer suitable for use in the compositions described herein is disclosed in Shah, U.S. Pat. Nos. 5,814,329 and 5,942,243, which are incorporated herein by reference in their entireties. The copolymer has a hydrophilic polymeric main chain and a hydrophobic polystyrene graft chain (FIG. 1). The main chain comprises monomeric units having acidic groups and optionally neutral monomeric units. The graft copolymer is prepared by free radical initiated polymerization of a polystyrene macromonomer having an ethylenically, unsaturated functional group (FIG. 2) with the acidic and neutral hydrophilic comonomers. The acidic comonomers suitable for preparation of the graft copolymer include but are not limited to acrylic acid, methacrylic acid, itaconic acid, 2-acrylamido-2-methyl-propane sulfonic acid, 2-sulfoethyl methacrylate, and vinyl phosphonic acid. The neutral comonomers of the main chain may include without limitation acrylamide, methacrylamide, 2-hydroxyethyl methacrylate, N,N-dimethylacrylamide, polyethylene glycol monomethacrylate, and glyceryl methacrylate. A particularly useful bioadhesive graft copolymer for use in the transdermal composition is poly(N,N-dimethylacrylamide-co-acrylic acid-co-polystyrene ethyl methacrylate) (FIG. 3), which is identified by its CAS No. 547763-79-1 and is commercially available under the trade name PHARMADUR® (Polytherapeutics, Inc.). The graft copolymer may be present in the transdermal composition in an amount ranging from about 1% to about 10%, by weight of the total composition. Suitably, the graft copolymer is in an amount ranging from about 2% to about 8%, or about 2% to about 6%, or about 2%, about 3%, about 4%, about 5%, about 6%, about 7% or about 8%, by weight of the total composition.

Pharmaceutically acceptable dosage forms of the transdermal compositions of the present invention include without limitation all conventional forms such as solution, gel, emulsion, suspension, or an ointment. The dosage form of the transdermal composition upon application to skin is designed to dry and form a bioadherent polymeric film incorporating the drug, the skin permeation enhancer, and non-volatile excipients of the composition. The drug is delivered to the body from the polymeric film by a process of diffusion through the skin. Different excipients may be used in the transdermal composition depending on the kind of dosage form to be prepared. Various excipients that may be used include solvent for the drug, thickener, preservative, antioxidant, chelating agent, fragrance, other agents such as emulsifier, emollient, waxes etc. The dosage form is formulated with emphasis on its spreadability on skin, drying time, cosmetic feel, and the user acceptability. Excipients to be used in the composition of this invention should be non-toxic and non-irritating and non-sensitizing to skin even upon repeated application to the same skin site.

A clear solution or a gel is a preferred dosage form for the transdermal composition. A clear solution or a gel by definition is a homogeneous composition in which all the ingredients are in a dissolved state. The NSAIDs for use in this invention have a limited solubility in water. Therefore, an alcoholic or a hydroalcoholic vehicle is more appropriate for the preparation of a solution or a gel. Lower alkyl alcohols, particularly ethanol and isopropyl alcohol, are preferred solvents for use in this invention. The proportion of the alcohol in the composition may vary in amount ranging from about 10% to about 50% by weight of the total composition. The drug solubility in the composition is generally optimum at a pH of about 7 to 8. Therefore, a pH of the solution in this range is preferred. Bases such as sodium hydroxide, sodium bicarbonate, ammonia, and diethyl amine may be used for adjusting the pH of the composition. Depending upon the desired viscosity of the composition, optionally hydrophilic polymers such as hydroxyethyl cellulose, carbomer, or hydropropyl methyl cellulose, poly(vinyl pyrrolidone), and poly(vinyl alcohol) may also be incorporated in the composition as a thickener. The solution or gel composition may include one or more antioxidants and chelating agents for stabilizing the drug from oxidative degradation. Common pharmaceutically acceptable antioxidants include ascorbic acid, ascorbyl palmitate, alpha tocopherol, butylated hydroxyanisole, butylated hydroxytoluene, monothioglycerol, propyl gallate, sodium ascorbate, and sodium metabisulfite. Suitable chelating agents include ethylene diamine tetraacetic acid (EDTA), edetic acid, citric acid monohydrate, disodium edetate, trisodium edetate, fumaric acid, malic acid, and tartaric acid, gluconic acid, and cyclodextrin.

A typical method of preparation of a clear transdermal solution/gel comprises first dissolving the bioadhesive graft copolymer and optionally a thickener polymer under stirring in a mixture of water, an alcohol solvent, and the skin permeation enhancer. Then the drug is added in small increments to the solution under continuous stirring. Upon dissolution of the drug in the solution, pH of the resultant mixture is carefully adjusted within the range of 7 to 8 to form a transdermal composition of the present invention.

Preparation of creams and lotions (oil-in-water emulsion) and ointments (water-in-oil emulsions) is well known to those skilled in the art. For example, excipients for use in the preparation of such pharmaceutical emulsion compositions comprising diclofenac or its salt and their methods of preparation are disclosed by Fankhauser (U.S. Pat. No. 4,999,379), which is incorporated herein by reference in its entirety. Key excipients for the preparation of such emulsions include oils, waxes, emulsifiers and emollients.

It was unexpectedly and surprisingly found that the transdermal compositions disclosed herein, incorporating a bioadhesive graft copolymer, provide a significantly greater enhancement in skin permeation rate of an NSAID than has been known previously. For example, Kisak, et al. (U.S. Patent Application Publication No. 2008/0300311) have disclosed that by formulating a thickener in a diclofenac gel solution consisting of skin permeation enhancer DMSO, propylene glycol, ethanol, glycerol, and water, an increase in the flux of diclofenac was observed compared to the comparator formulation without the thickener. Various thickeners, which were tested, included CARBOPOL®, hydroxyethyl cellulose, hydroxypropyl cellulose, hydropropyl methyl cellulose, guar gum, poly(vinyl pyrrolidone), POLOXAMER®, and ULTREZ® polymer (Noveon). The highest increase in the diclofenac flux was obtained with CARBOPOL®. However, the CARBOPOL® thickened formulations only provided from 1 to 5-fold increase in diclofenac flux as compared to the comparator formulation without the thickener (Table 1).

TABLE 1
Skin Permeation Enhancement of Diclofenac in Kisak Gels

| | | Test | | | | |
|---|---|---|---|---|---|---|
| | | Sample | Diclofenac Flux J, mcg/cm²/hr | | | |
| Formulation | Thickener, | Weight, | | | J/Jcomparator* | |
| I.D. | % | mg | J/24 hr | J/48 hr | 24 Hr | 48 Hr |
| Comparator* | | 15 | 0.125 | 0.17 | | |
| PP54 | Carbopol | 15 | 0.35 | 0.73 | 2.8 | 4.3 |
| | 971/1 | | | | | |
| Comparator* | | 200 | 1.46 | 2.08 | | |
| P981a | Carbopol | 200 | 3.75 | 5.83 | 2.6 | 2.8 |
| | 981/0.9 | | | | | |
| Comparator* | | 50 | 0.625 | 0.63 | | |
| PP52 | Carbopol | 50 | 2.5 | 3.7 | 4 | 5.9 |
| | 971/1 | | | | | |
| Comparator* | | 20 | 0.625 | 1 | | |
| F971 | Carbopol | 20 | 2.29 | 3.54 | 3.7 | 3.5 |
| | 971/1.1 | | | | | |

*COMPARATOR formulation is as shown herein in Example 3.

It can be seen from the examples shown below (see Example 3 & Table 6) that the transdermal compositions disclosed herein formulated with diclofenac and a bioadhesive graft copolymer (PHARMADUR®) and lower concentrations of the same skin permeation enhancer DMSO as the Kisak, et al.'s comparator composition provide a diclofenac flux enhancement in the range of 15 to 28-fold over the comparator composition. The large magnitude of this enhancement of diclofenac flux is totally unexpected. B. Michniak, et al. (“Evaluation of Novel Polymer for Transdermal Permeation of Caffeine in Presence of Different Concentrations of Chemical Enhancer”, Bozena Michniak, R. Thakur, and K. R. Shah, Poster #323, Proceedings of Controlled Release Society 32nd Annual Meeting, Miami Beach, Fla., June 2005.) have previously reported that aqueous formulations comprising caffeine (drug), oleic acid as enhancer, and PHARMADUR® graft copolymer exhibit about 1 fold increase in the skin permeation rate (human cadaver skin) of caffeine as compared to control formulations without the PHARMADUR® graft copolymer. The magnitude of this increase in the flux of caffeine by the use of bioadhesive graft copolymer is in the range of that disclosed by Kisak, et al. in the patent application cited above. Thus, the very large magnitude of enhancement of diclofenac flux by the transdermal compositions disclosed herein is surprising and unexpected and is unique to the specific combinations of the NSAID, skin permeation enhancer, and the bioadhesive graft copolymer within their specified proportions as described herein.

The transdermal NSAID compositions of the present invention are suitably used for relief of inflammation and pain due to arthritis and muscular or muscoskeletal injuries. The dosage of the composition to be applied will depend on whether local or systemic therapeutic effect is desired. For local therapeutic effect, the amount of the dose would be proportional to the surface area of the skin where the drug's pharmacological activity is required. For systemic therapeutic effect, about 1.5 to 3.0 g. of the transdermal composition, having a drug concentration of about 3% to about 7%, is spread over a 300 to 600 sq. cm of skin surface, which need not be the affected site of the body. Typically this skin surface could be on legs, arms, abdomen, or back as may be convenient to the user. An invisible bioadherent polymeric film containing the drug, skin permeation enhancer, and non-volatile excipients is formed on the skin upon application of the transdermal composition. It has been disclosed previously that such a graft copolymer film, as exemplified with properties of the PHARMADUR® polymer, is retained on the skin for a prolonged period of time and can provide sustained release of drug for an extended period of time. [See “PharmaDur® Bioadhesive Delivery System”, K. R. Shah in John J. Wille, ed. “Skin Delivery Systems; Transdermals, Dermatologicals, and Cosmetic Actives”, Blackwell Publishing, Ames, Iowa, 2006, p 211-222.] Thus, the dried transdermal composition functions as a “virtual patch” without the limitations of plastic patches. Typically once/day or twice/day application of the dose of the transdermal composition are utilized. Transdermally administered drug is absorbed continuously over a prolonged period of time in contrast with a relatively rapid absorption through the GI tract. Thus, sustained lower but effective concentrations of the drug are available in the blood circulation by transdermal administration of the drug.

In embodiments, compositions for transdermal administration of one or more non-steroidal anti-inflammatory drugs (NSAIDs) to an animal are provided. The compositions can provide either a systemic or a local therapeutic effect.

In suitable embodiments, the compositions comprise about 1% to about 10% of a film forming hydrophilic graft copolymer, as described in U.S. Pat. Nos. 5,814,329 and 5,942,243, the disclosures of which are incorporated by reference herein in their entireties. Suitably, the graft copolymer is a reaction product of a polystyrene macromonomer having an ethylenically unsaturated functional group and at least one hydrophilic acidic monomer having an ethylenically unsaturated functional group. Suitably, the weight percent of the polystyrene macromonomer in the graft copolymer is between about 1 and about 20%, and the weight percent of the total hydrophilic monomer in the graft copolymer is between 80 and 99%, wherein at least about 10% of said total hydrophilic monomer is acidic, the graft copolymer when fully hydrated having an equilibrium water content of at least 90%. Suitably, the graft copolymer is present in the composition in an amount sufficient to cause the composition to form a water swollen but insoluble jelly like mass upon contact with a biological environment (e.g., skin, mucosal tissue, muscle, bone, cells, etc.).

In embodiments, the compositions comprise about 0.1% to about 10% of one or more non-steroidal anti-inflammatory drugs (NSAID). Suitable NSAIDs for use in the compositions are described herein. Suitably, the compositions comprise about 2% to about 50% of a skin permeation enhancer. In embodiments, the skin permeation enhancer is 2-pyrollidone, N-methyl 2-pyrrolidone (NMP) N-octyl 2-pyrrolidone, N-dodecyl-2-pyrrolidone, N-(2-hydroxyethyl)-2-pyrrolidone, dimethyl sulfoxide (DMSO), decylmethyl sulfoxide, or a mixture of two or more of these enhancers.

Suitably, the compositions described herein exhibit a skin permeation rate of the non-steroidal anti-inflammatory drug that is at least five times greater than the skin permeation rate of the non-steroidal anti-inflammatory drug in a control composition that does not comprise the graft copolymer. As described throughout and in the Examples, when the skin permeation rate of the NSAID contained in the compositions of the invention is compared to the skin permeation rate of the NSAID in a control composition that does not comprise the graft copolymer, the permeation rate is surprisingly and unexpected increased by at least five times, or even by at least 10 times, at least 15 times, at least 20 times, at least 25 times or even at least 30 times, greater.

As described throughout, for comparison to the compositions of the invention, suitable control compositions that do not comprise a graft copolymer are used. Exemplary such control compositions include (i) VOLTAREN® Gel (Novartis Consumer Health, Inc.) Ingredients—1% diclofenac sodium, carbomer homopolymer Type C, cocoyl caprylocaprate, fragrance, isopropyl alcohol, mineral oil, polyoxyl 20 cetostearyl ether, propylene glycol, purified water, and strong ammonia solution, and (ii) PENNSAID® (Nuvo Research Inc.) Ingredients: 1.5% diclofenac sodium, 45.5% dimethyl sulfoxide, ethanol, glycerine, propylene glycol, and purified water.

In suitable embodiments, the NSAID in the compositions is diclofenac sodium. In embodiments, the skin permeation enhancer is N-methyl 2-pyrrolidone, dimethyl sulfoxide, or decylmethyl sulfoxide, and suitably the skin permeation enhancer is a mixture of N-methyl 2-pyrrolidone and dimethyl sulfoxide at a weight:weight ratio of about 1:3 to about 3:1 (N-methyl 2-pyrrolidone:dimethyl sulfoxide).

Suitably, the graft copolymer is poly(N,N-dimethylacrylamide-co-acrylic acid co-polystyrene ethyl methacrylate), and in embodiments is present at about 2% to about 6%. In embodiments, the NSAID is present at a about 0.5% to about 5%, and in further embodiments, the skin permeation enhancer is present at about 5% to about 40%.

In further embodiments, compositions for transdermal administration of one or more non-steroidal anti-inflammatory drugs to an animal for systemic or local therapeutic effect are provided. Suitably, the compositions comprise about 1% to about 10% of a film forming hydrophilic graft copolymer comprising poly(N,N-dimethylacrylamide-co-acrylic acid-co-polystyrene ethyl methacrylate), about 0.1% to about 10% of diclofenac or a salt of diclofenac; and about 2% to about 50% of a skin permeation enhancer comprising a mixture of N-methyl 2-pyrrolidone (NMP) and dimethyl sulfoxide (DMSO) at a weight:weight ratio of about 1:3 to about 3:1 (N-methyl 2-pyrrolidone:dimethyl sulfoxide). Suitably, the compositions exhibit a skin permeation rate of the diclofenac or salt of diclofenac that is at least five times greater than the skin permeation rate of the diclofenac or salt of diclofenac in the same composition that does not comprise the graft copolymer.

In suitable embodiments, the graft copolymer is present at about 2% to about 3%, the diclofenac or salt of diclofenac is present at about 4% to about 6% and the skin permeation enhancer is present at about 10-20% N-methyl 2-pyrrolidone (NMP) and 20-30% dimethyl sulfoxide (DMSO). In still further embodiments, the graft copolymer is present at about 2.5% to about 4%, the diclofenac or salt of diclofenac is present at about 2% to about 3% and the skin permeation enhancer is present at about 5-10% N-methyl 2-pyrrolidone (NMP) and 5%-10% dimethyl sulfoxide (DMSO).

The transdermal compositions disclosed herein provide the following advantages:
Provide sustained therapeutic effect.
Minimize irritation of the gastrointestinal (G) tract. Since the NSAID is delivered transdermally, it bypasses the GI tract.
Lower systemic toxicity risks by avoiding high peak drug concentrations that are found in blood by its oral administration,
Lower liver toxicity risk by avoiding hepatic first pass metabolism.

The transdermal compositions of this invention may be packaged in any convenient container, which include without limitation tube, dropper bottle, unit dose packet, and metered multi-dose pump dispenser.

Another embodiment of the present invention is to provide a method of transdermal administration of NSAID, for either local or systemic therapeutic effect, by topical application to skin of the transdermal composition described herein. The methods can also be used to increase the skin permeation rate of an NSAID.

Thus, in embodiments, methods of transdermally administering one or more non-steroidal anti-inflammatory drugs to an animal for systemic or local therapeutic effect are provided. Such methods suitably comprise applying a composition as described herein to the skin of the animal.

Suitably, the compositions comprise about 1% to about 10% of a film forming hydrophilic graft copolymer, the graft copolymer being a reaction product of: a polystyrene macromonomer having an ethylenically unsaturated functional group; and at least one hydrophilic acidic monomer having an ethylenically unsaturated functional group. Suitably, the weight percent of the polystyrene macromonomer in the graft copolymer is between about 1 and about 20%, and the weight percent of the total hydrophilic monomer in the graft copolymer is between 80 and 99%, wherein at least about 10% of the total hydrophilic monomer is acidic, the graft copolymer when fully hydrated having an equilibrium water content of at least 90%. Suitably the graft copolymer is present in the composition in an amount sufficient to cause the composition to form a water swollen but insoluble jelly like mass upon contact with a biological environment.

The compositions suitably comprise about 0.1% to about 10% of one or more non-steroidal anti-inflammatory drugs (NSAID) and about 2% to about 50% of a skin permeation enhancer selected from the group consisting of 2-pyrollidone, N-methyl 2-pyrrolidone (NMP), N-octyl 2-pyrrolidone, N-dodecyl-2-pyrrolidone, N-(2-hydroxyethyl)-2-pyrrolidone, dimethyl sulfoxide (DMSO), decylmethyl sulfoxide, and mixtures thereof.

The methods further comprise allowing the composition to dry by evaporation of volatile excipients.

Suitably, the compositions exhibit a skin permeation rate of the non-steroidal anti-inflammatory drug that is at least five times greater than the skin permeation rate of the non-steroidal anti-inflammatory drug in the same composition that does not comprise the graft copolymer.

In embodiments, the composition comprises about 1% to about 10% of a graft copolymer comprising poly(N,N-dimethylacrylamide-co-acrylic acid-co-polystyrene ethyl methacrylate), about 0.1% to about 10% of diclofenac or a salt of diclofenac; and about 2% to about 50% of a skin permeation enhancer comprising a mixture of N-methyl 2-pyrrolidone (NMP) and dimethyl sulfoxide (DMSO) at a weight:weight ratio of about 1:3 to about 3:1 (N-methyl 2-pyrrolidone:dimethyl sulfoxide).

Suitably, the compositions are administered at least one a day (e.g., one a day, twice a day, three times a day, etc.) for as long as desired, suitably on the order of days to weeks to months, or longer if desired. The compositions can be administered to any skin surface, including the hand, arms, trunk, back, legs, feet, etc.

Also provided are methods of treating inflammation and pain in an animal. Suitably, such methods comprise applying the compositions disclosed herein to the skin of an animal in need of such treatment. As described herein, the methods can suitably be used for systemic treatment of inflammation and pain, or can be used for local treatment of inflammation and pain. In embodiments where the methods are for treatment of systemic treatment, generally higher amounts of the NDSAID, permeation enhancers and graft copolymers are used, as described throughout.

Suitably, the compositions are administered at least one a day (e.g., one a day, twice a day, three times a day, etc.) for as long as desired, suitably on the order of days to weeks to months, or longer if desired. The compositions can be administered to any skin surface, including the hand, arms, trunk, back, legs, feet, etc.

It will be readily apparent to one of ordinary skill in the relevant arts that other suitable modifications and adaptations to the methods and applications described herein can be made without departing from the scope of any of the embodiments. The following examples are included herewith for purposes of illustration only and are not intended to be limiting.

### EXAMPLES

### Materials

(a) Bioadhesive Graft Copolymer: Poly(N,N-dimethylacrylamide-co-acrylic acid-co-polystyrene ethyl methacrylate) (PHARMADUR® polymer, Polytherapeutics, Inc.),
(b) NSAID: diclofenac sodium USP (Spectrum Chemicals),
(c) Skin Permeation Enhancers: N-methyl 2-pyrrolidone (PHARMASOLVE®, International Specialty Products), dimethyl sulfoxide USP (Gaylord Chemicals), dimethyl isosorbide (ARLASOLVE®DMI, Croda Inc.), ethoxydiglycol (TRANSCUTOL® CG, Gottefosse)
(d) Solvents: Isopropyl alcohol USP (Spectrum Chemicals) and distilled water.
(e) Comparative or Control Formulations/Products: (i) VOLTAREN® Gel (Novartis Consumer Health, Inc.) Ingredients—1% diclofenac sodium, carbomer homopolymer Type C, cocoyl caprylocaprate, fragrance, isopropyl alcohol, mineral oil, polyoxyl 20 cetostearyl ether, propylene glycol, purified water, and strong ammonia solution. (ii) PENNSAID® (Nuvo Research Inc.) Ingredients: 1.5% diclofenac sodium, 45.5% dimethyl sulfoxide, ethanol, glycerine, propylene glycol, and purified water.
Method of Preparation of Test Formulations: The method described below was used to make all test formulations of the transdermal composition.

First a 500 g. stock solution containing 6.5% PHARMADUR® polymer by weight in distilled water was prepared by stirring granulated form of the polymer in water for about 2-3 hours at 60° C. Batch size for each of the transdermal formulation was 20 g. In a 50-ml beaker containing a magnetic stirrer were placed 8.45 g. of the 6.5% PHARMADUR® solution, and the required calculated amounts of the enhancer, water, and isopropyl alcohol. The mixture was allowed to stir for about 10 minutes to form a homogenous clear solution. Then 1 g. of diclofenac sodium was added in small increments under vigorous stirring. The mixture was then allowed to stir for an additional 15 minutes to ensure complete dissolution of the drug. Then the pH of the resultant clear and slightly viscous solution was adjusted within the range of 7.1 to 7.6 by addition of few drops of 10% ammonium hydroxide solution in water.

Skin Permeation Study Method:

Study of skin permeation of diclofenac from test and control formulations was done using human cadaver skin and Vertical Franz Diffusion Cells (receptor volume 5.2 ml, Permegear, Inc., Bethlehem, Pa.) having a donor cross-sectional area of 0.64 cm². The cadaver skin (posterior leg) was obtained from New York fire Fighters Skin Bank, New York, N.Y. and stored under dry ice. At the time of experiment, skin samples were cut into appropriate sized pieces, slowly thawed and hydrated in phosphate buffer saline and then mounted on Franz diffusion cell. Each cell contained Phosphate Buffer Saline (PBS), pH 7.4 that was maintained at 37° C. and stirred continuously at 600 rpm using a magnetic stirrer. Five replicates of each formulation were tested. At time zero, 50 microliters (μl) of each of the formulations was added to the donor compartment of Franz diffusion cell. VOLTAREN® Gel (Novartis) was used as a control in every study using about 81±1 mg of VOLTAREN®gel in the corresponding donor compartment of Franz cells. All the donor compartments were left uncovered for uniform drying of the formulation on skin surface. A 300-μl sample of the receptor fluid from every Franz cell was removed through the cell sampling port and immediately replaced with an equivalent volume of PBS, pH 7.4. Receptor aliquots were withdrawn at intervals of 0, 2, 4, 6, 8, 12, 24, 28 and 32 hr. All the samples were then analyzed by high-pressure liquid chromatography (HPLC) with ultraviolet detection for concentration of diclofenac in the samples. Analysis by HPLC and subsequent calculations yielded penetration parameters that included flux (J), lag time, and amount of the drug penetrated into the receptor per unit area of skin in 32 hours (Q₃₂).

### Example 1

Diclofenac flux for VOLTAREN® Gel through 10 different donor skins is summarized in Table 2.

TABLE 2
Diclofenac Flux for VOLTAREN ® Gel

| | Diclofenac J |
|---|---|
| Donor Skin # | mcg/cm²/hr |
| 1 | 0.6 |
| 2 | 1.2 |
| 3 | 1.6 |
| 4 | 0.4 |
| 5 | 0.5 |
| 6 | 2.0 |
| 7 | 1.3 |
| 8 | 1.2 |
| 9 | 0.5 |
| 10 | 2.3 |
| AVERAGE | 1.16 |

Since there is a wide degree of subject to subject variations in skin permeation of drugs in human population, an average value of the flux may be regarded as a reasonable estimation of the product's performance in human use. Therefore, in subsequent evaluation of the test compositions of this invention, different flux values for different formulations with different donor skins are compared with one another after normalization of their flux with the 10-donor skin average flux (J=1.16 mcg/cm²/hr) for VOLTAREN® Gel as expressed by the following equation:

Normalized diclofenac flux, J for the Test Formulation and donor skin #N=Actual J_{N} for Test Formulation×Average J for Voltaren® Gel/Actual J_{N} for VOLTAREN® Gel

=Actual J_{N} for Test Formulation×1.16/Actual J_{N} for VOLTAREN® Gel

VOLTAREN®Gel serves as a suitable benchmark and a control for correlating the performance of the test formulations. Therefore, the normalized diclofenac flux is a better predictor of performance of the compositions of this invention in human use.

### Example 2

Formulation ingredients and their concentrations in the transdermal compositions, which were tested for their skin permeation rate measurement, are shown in Table 4. The results of their skin, permeation study are shown in Table 5.

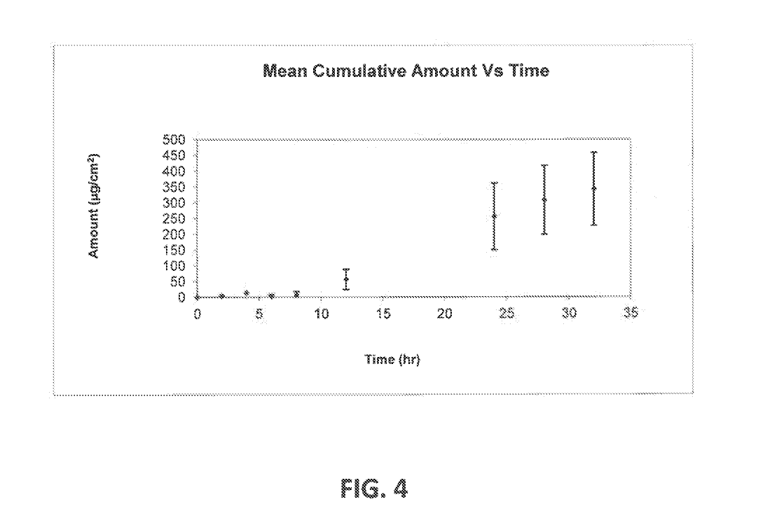

Skin permeation parameters for all of the formulations were determined as illustrated below for the formulation PG-11. The cumulative amount of diclofenac that permeated through the skin was measured (see Table 3) and a plot of cumulate amount permeated versus time was generated, as shown in FIG. 4.

TABLE 3
Diclofenac Skin Permeation

| | Cumulative Amount | | |
|---|---|---|---|
| | Permeated | | Std Error |
| Time (hr) | Mean, mcg/sq. cm/hr | Std Dev | for plotting |
| 0 | 0.00 | 0.00 | 0.00 |
| 2 | 4.13 | 0.00 | 0.00 |
| 4 | 13.88 | 0.00 | 0.00 |
| 6 | 3.08 | 5.75 | 2.57 |
| 8 | 7.43 | 10.03 | 4.49 |
| 12 | 56.67 | 32.18 | 14.39 |
| 24 | 255.41 | 105.88 | 47.35 |
| 28 | 307.60 | 109.21 | 48.84 |
| 32 | 342.49 | 115.25 | 51.54 |

To determine the flux, J, the slope of best statistical linear fit of the steady state (6 to 32 hours) portion of the skin permeation data shown in FIG. 4 was taken. This provided a value of 14.0 mcg/sq. cm/hr. The lag time was determined as the period prior to the attainment of the steady state diclofenac permeation, approximately 4 hours. Q32 was then determined as the amount of diclofenac permeated per unit area in 32 hours, at 342.5 mcg/sq.cm.

TABLE 4
Transdermal Test Compositions

| Formulation | PHARMADUR ® | Diclofenac, | | Isopropyl | | |
|---|---|---|---|---|---|---|
| I.D. | Polymer, % | Sodium, % | Enhancer % | Alcohol, % | Water, % | pH |
| PG-1 | 2.85 | 5 | DMI¹, 20 | 30 | 42.15 | 7.1 |
| PG-2 | 2.85 | 5 | EDG², 20 | 30 | 42.15 | 7.1 |
| PG-3 | 2.85 | 5 | NMP³, 20 | 30 | 42.15 | 7.1 |
| PG-4 | 2.85 | 5 | NMP, 30 | 22 | 40.15 | 7.5 |
| PG-5 | 2.85 | 5 | NMP, 40 | 12 | 40.15 | 7.5 |
| PG-6 | 2.85 | 5 | DMSO⁴, 20 | 30 | 42.15 | 7.3 |
| PG-7 | 2.85 | 5 | DMSO, 30 | 22 | 40.15 | 7.3 |
| PG-8 | 2.85 | 5 | DMSO, 40 | 12 | 40.15 | 7.4 |
| PG-9 | 2.85 | 5 | NMP, 10 | 22 | 40.15 | 7.3 |
| | | | DMSO, 20 | | | |
| PG-10 | 2.85 | 5 | NMP, 10 | 12 | 40.15 | 7.5 |
| | | | DMSO, 30 | | | |
| PG-11 | 2.85 | 5 | NMP, 15 | 12 | 40.15 | 7.6 |
| | | | DMSO, 25 | | | |
| PG-12 | 2.85 | 5 | NMP, 20 | 12 | 40.15 | 7.4 |
| | | | DMSO, 20 | | | |
| PG-13 | 2.85 | 5 | NMP, 25 | 12 | 40.15 | 7.5 |
| | | | DMSO, 15 | | | |

¹Dimethyl Isosorbide,
²Ethoxydiglycol,
³N-Methyl 2-Pyrrolidone,
⁴Dimethyl Sulfoxide

TABLE 5
Skin Permeation Results of Transdermal Test Compositions

| | | | Test Formulation | | VOLTAREN ® | | Test |
|---|---|---|---|---|---|---|---|
| Test | | | Diclofenac | | Diclofenac | | Formulation |
| Formulation | Donor | | J | Q*₃₂ | J | Q*₃₂ | Normalized J, |
| I.D. | Skin # | Enhancer % | mcg/cm²/hr | mcg/cm² | mcg/cm²/hr | mcg/cm² | mcg/cm²/hr |
| PG-1 | 1 | DMI | 0.1 | 2.1 | 0.6 | 13.0 | 0.19 |
| PG-2 | 1 | EDG | 0.4 | 10.8 | 0.6 | 13.0 | 0.77 |
| PG-3 | 1 | NMP, 20 | 4.2 | 107.8 | 0.6 | 13.0 | 8.1 |
| PG-4 | 2 | NMP, 30 | 9.9 | 263.2 | 1.2 | 36.4 | 9.6 |
| PG-5 | 2 | NMP, 40 | 12.5 | 318.2 | 1.2 | 36.4 | 12.1 |
| PG-6 | 1 | DMSO, 20 | 2.5 | 61.0 | 0.6 | 13.0 | 4.8 |
| PG-7 | 2 | DMSO, 30 | 7.7 | 196.7 | 1.2 | 36.4 | 7.4 |
| PG-8 | 2 | DMSO, 40 | 8.8 | 230.8 | 1.2 | 36.4 | 8.5 |
| PG-9 | 3 | NMP, 10 | 11.5 | 313.0 | 1.6 | 39.4 | 8.4 |
| | | DMSO, 20 | | | | | |
| PG-10 | 3 | NMP, 10 | 15.2 | 386.9 | 1.6 | 39.4 | 11.0 |
| | | DMSO, 30 | | | | | |
| PG-11 | 7 | NMP, 15 | 14.0 | 342.5 | 1.3 | 37.3 | 12.5 |
| | | DMSO, 25 | | | | | |
| PG-12 | 3 | NMP, 20 | 13.2 | 358.0 | 1.6 | 39.4 | 9.6 |
| | | DMSO, 20 | | | | | |
| PG-13 | 3 | NMP, 25 | 13.1 | 366.5 | 1.6 | 39.4 | 9.5 |
| | | DMSO, 15 | | | | | |

*Q₃₂—Cumulative amount of diclofenac permeated through unit area of skin into the receptor fluid in 32 hours.

The study results shown in Table 5 indicate that the only NMP and DMSO provide a strong enhancement in the flux (J) and cumulative amount (Q₂₃) of transdermal diclofenac from formulations with the bioadhesive graft copolymer. Flux and Q₃₂ values for the DMI and EDG formulations, PG-1 and PG-2 respectively, are lower than those for the VOLTAREN® Gel control. The above results of the present invention also indicate that, by the use of mixtures of NMP and DMSO in the transdermal compositions, higher flux of diclofenac can be obtained.

Steady state concentration of diclofenac in blood from transdermal administration of the compositions of this invention to a human body can be calculated as follows from the flux measurements obtained above using human cadaver skin:

Diclofenac Flux=J mcg/sq. cm/hr

For 400 sq cm skin surface of application of the transdermal composition,

$$\begin{matrix}Diclofenac Daily Dose = J \times 400 \times 24 mcg \text{/} day \\ = 9600 \times J mcg \text{/} day\end{matrix}$$

Diclofenac Clearance, Cl=252 ml/hr/kg
(Reference: “Goodman & Gilman's The Pharmacological Basic of Therapeutics”)
Steady State Diclofenac Blood Concentration, C

$$\begin{matrix}C = Diclofenac Daily Dose \text{/} Cl \\ = \frac{9600 \times J mcg}{24 hr} \times \frac{hr \times kg}{252 ml} \\ = 1.587 \times J mcg \cdot Kg \text{/} ml\end{matrix}$$

For 70 Kg subject,

$$\begin{matrix}C = \frac{1.587 \times J mcg \cdot Kg}{ml \times 70 Kg} \\ = 22.67 \times J ng \text{/} ml\end{matrix}$$

PG-6 composition exhibited the lowest J=4.8 mcg/cm²/hr.
Calculated C=4.8×22.67=109 ng/ml
PG-11 composition exhibited the highest J=12.5 mcg/cm²/hr.
Calculated C=12.5×22.67=283 ng/mil
Minimum effective diclofenac blood concentration=50 ng/ml
[Ref. Toshiaki Nishihata, et al. Int. Pharmaceutics, 42, 251 (1988)]

Thus, drug concentrations in blood, calculated from in vitro cadaver skin permeation data, of the above tested transdermal compositions of this invention are well within the range of effective drug levels. It is well established in the scientific field that a good correlation exists between in vitro and human in vivo performance of transdermal formulations when the in vitro data are obtained using human cadaver skin. [Reference: T. J. Franz, et al., Skin Pharmacology and Physiology, 22, 276-286 (2009)] The reason for such correlation is that the stratum corneum of skin, which is the actual rate-controlling barrier for transdermal drug delivery, is the same in both cadaver and living human subjects. Therefore, the transdermal compositions of this invention can provide systemic therapeutic effect. When only local therapeutic effect is desired, a lower concentration of the enhancer should be used in the transdermal composition. For example, a suitable diclofenac composition for only local therapeutic effect can be comprised of 2% to 3% diclofenac sodium, 2.5% to 4% bioadhesive graft copolymer, and 5% to 10% of NMP or DMSO enhancer or combinations thereof. Thus, the transdermal composition of this invention can provide a local therapeutic effect at a much lower enhancer concentration than that present in formulations such as PENNSAID® product (Example 3, Table 6).

### Example 3

In this example the skin permeation of the transdermal compositions of this invention are compared with those of the Kisak, et al. patent application referenced above. As disclosed therein, addition of a thickener to the basic comparator formulation results in 1 to 5-fold increase in diclofenac flux, as compared to the comparator formulation without the thickener. Their comparator formulation appears to be the same as their marketed product PENNSAID® as shown below. Therefore, the transdermal formulations of the present invention are compared to the commercially available PENNSAID® to confirm that the remarkably high diclofenac flux values are due to the bioadhesive graft copolymer.
PENNSAID® Formulation/Product Label: 15% diclofenac sodium, 45.5% dimethyl sulfoxide, propylene glycol, ethanol, glycerine, and purified water.
Kisak, et al. Comparator Formulation: 1.5% diclofenac sodium, 45.5% dimethyl sulfoxide, 11.2% propylene glycol, 11.79% ethanol, 11.2% glycerine, 18.81% water.
   Concentrations of key formulation ingredients of the transdermal compositions and comparison of their flux J with that of PENNSAID® are shown in Table 6.

TABLE 6
Comparison of Flux of the Transdermal Compositions with PENNSAID ®

| | | | | | Normalized | |
|---|---|---|---|---|---|---|
| Formulation | | | Diclofenac J, mcg/cm²/hr | | J, | J_{Formulation} |
| I.D. | Diclofenac % | Enhancer % | Formulation | VOLTAREN ® | mcg/cm²/hr | J_{Pennsaid} |
| Pennsaid ® | 1.5 | DMSO, 45 | 0.1 | 0.4 | 0.29 | 1 |
| PG-6 | 5 | DMSO, 20 | 2.5 | 0.6 | 4.8 | 16.5 |
| PG-7 | 5 | DMSO, 30 | 7.7 | 1.2 | 7.5 | 25.9 |
| PG-8 | 5 | DMSO, 40 | 8.8 | 1.2 | 8.5 | 29.3 |
| PG-4 | 5 | NMP | 9.9 | 1.2 | 9.6 | |
| PG-14* | 5 | 30 | 7.8 | 0.4 | 22.6 | |
| AVERAGE | | | | | 16.1 | 55 |
| PG-15* | 1.5 | NMP | 4.9 | 0.4 | 14.2 | 49 |
| | | 30 | | | | |

*Compositions of the excipients in PG-14 and PG-15 are same as that of PG-4.

The above results of skin permeation studies of the transdermal compositions (PG-6, PG-7, and PG-8) show that these compositions containing a lower concentration of the enhancer DMSO than the PENNSAID®product result in a significantly greater flux than PENNSAID®. The flux of the composition with 30% NMP as the enhancer with 5% drug (PG-4 and PG-14) is also comparable with the flux of the composition with the same amount of NMP and 1.5% drug (PG-15). Thus, the enhancer concentration and not the drug concentration was found to affect diclofenac flux from bioadhesive graft copolymer film formed on skin by the transdermal composition of this invention. Therefore, the tremendously greater flux of diclofenac obtained from the above compositions compared to that from PENNSAID®cannot be ascribed to the difference in the drug concentration. The magnitude of increase in the drug flux can be inferred to be due to the effect of the bioadhesive graft copolymer film on skin. The magnitude of enhancement in flux of the diclofenac obtained from the transdermal compositions of the present invention (15 to 28-fold increase) is very much larger than those of the Kisak compositions (1 to 5-fold increase), which incorporate other thickener polymers.

### Example 4

The composition of PG-11 (Example 2, Table 4) was tested by treatment for an osteoarthritis patient periodically for 4+ months to assess its therapeutic efficacy in mitigating pain and potential for any adverse skin reactions.

Frequency of dose application was once/day, mainly after showering. The formed polymer film on skin was washed away next day during showering. No oral anti-inflammatory drugs were administered during the period of treatment with the PG-11 composition.

In order to assess local therapeutic effect, about 250 mg of the formulation was applied to skin over and around each knee joint (both the legs), about 100 mg of PG-11 on right leg just below knee over outer tendon and muscle (˜10 cm×4 cm area), and 50 mg of PG-11 over both sides of the right wrist and uniformly spread by fingers. The patient experienced pain relief within 1 hour of treatment with the formulation and it continued all day long until washed off during showering.

In order to assess systemic therapeutic effect, about 2.5 gm of the PG-1 formulation was applied over abdominal skin surface (˜25 cm×16 cm) and uniformly spread by fingers. Relief from pain and/or joint stiffness (especially the knees) was perceived within 2 to 2.5 hours of treatment with the formulation and it continued all day long until washed off during showering. The relief of joint stiffness was perceived to be significantly greater than that resulting from oral single dose of 400 mg of ibuprofen (ADVIL®), which the patient was previously accustomed to use for relief of pain and joint stiffness.

No adverse skin reactions (itching, burning, erythema, or edema) were observed at any time on any skin area during all of the treatments with PG-11.

The results of skin permeation studies shown in the Examples 2, 3, and 4 illustrate very desirable transdermal performance of the disclosed compositions incorporating NSAID, specific enhancer, and the bioadhesive graft copolymer. There has thus been shown and described a novel composition for transdermal administration of non-steroidal anti-inflammatory drug which fulfills all the objects and advantages sought thereof.

It is to be understood that while certain embodiments have been illustrated and described herein, the claims are not to be limited to the specific forms or arrangement of parts described and shown. In the specification, there have been disclosed illustrative embodiments and, although specific terms are employed, they are used in a generic and descriptive sense only and not for purposes of limitation. Modifications and variations of the embodiments are possible in light of the above teachings. It is therefore to be understood that the embodiments may be practiced otherwise than as specifically described.

What is claimed is:

1. A composition for transdermal administration of one or more non-steroidal anti-inflammatory drugs to an animal for systemic or local therapeutic effect, the composition comprising:
a) about 1% to about 10% of a film forming hydrophilic graft copolymer, the graft copolymer being a reaction product of:
i. a polystyrene macromonomer having an ethylenically unsaturated functional group; and
ii. at least one hydrophilic acidic monomer having an ethylenically unsaturated functional group,
wherein the weight percent of the polystyrene macromonomer in the graft copolymer is between about 1 and about 20%, and the weight percent of the total hydrophilic monomer in the graft copolymer is between 80 and 99%, wherein at least about 10% of said total hydrophilic monomer is acidic, said graft copolymer when fully hydrated having an equilibrium water content of at least 90%,
the graft copolymer being present in the composition in an amount sufficient to cause the composition to form a water swollen but insoluble jelly like mass upon contact with a biological environment;
b) about 0.1% to about 10% of one or more non-steroidal anti-inflammatory drugs (NSAID); and
c) about 2% to about 50% of a skin permeation enhancer selected from the group consisting of 2-pyrollidone, N-methyl 2-pyrrolidone (NMP), N-octyl 2-pyrrolidone, N-dodecyl-2-pyrrolidone, N-(2-hydroxyethyl)-2-pyrrolidone, dimethyl sulfoxide (DMSO), decylmethyl sulfoxide, and mixtures thereof,
wherein the composition exhibits a skin permeation rate of the non-steroidal anti-inflammatory drug that is at least five times greater than the skin permeation rate of the non-steroidal anti-inflammatory drug in a control composition that does not comprise the graft copolymer.

2. The composition of claim 1, wherein the NSAID is diclofenac sodium.

3. The composition of claim 1, wherein the skin permeation enhancer is N-methyl 2-pyrrolidone.

4. The composition of claim 1, wherein the skin permeation enhancer is dimethyl sulfoxide.

5. The composition of claim 1, wherein the skin permeation enhancer is decylmethyl sulfoxide.

6. The composition of claim 1, wherein the skin permeation enhancer is a mixture of N-methyl 2-pyrrolidone and dimethyl sulfoxide at a weight:weight ratio of about 1:3 to about 3:1 (N-methyl 2-pyrrolidone:dimethyl sulfoxide).

7. The composition of claim 1, wherein the graft copolymer is poly(N,N-dimethylacrylamide-co-acrylic acid-co-polystyrene ethyl methacrylate).

8. The composition of claim 1, wherein the graft copolymer is present at about 2% to about 6%.

9. The composition of claim 1, wherein the NSAID is present at about 0.5% to about 5%.

10. The composition of claim 1, wherein the skin permeation enhancer is present at about 5% to about 40%.

11. The composition of claim 1, wherein the skin permeation rate of the non-steroidal anti-inflammatory drug is at least 10 times greater than the skin permeation rate of the non-steroidal anti-inflammatory drug in a control composition that does not comprise the graft copolymer.

12. The composition of claim 1, wherein the skin permeation rate of the non-steroidal anti-inflammatory drug is at least 20 times greater than the skin permeation rate of the non-steroidal anti-inflammatory drug in a control composition that does not comprise the graft copolymer.

13. A composition for transdermal administration of one or more non-steroidal anti-inflammatory drugs to an animal for systemic or local therapeutic effect, the composition comprising:
a) about 1% to about 10% of a film forming hydrophilic graft copolymer comprising poly(N,N-dimethylacrylamide-co-acrylic acid-co-polystyrene ethyl methacrylate);
b) about 0.1% to about 10% of diclofenac or a salt of diclofenac; and
c) about 2% to about 50% of a skin permeation enhancer comprising a mixture of N-methyl 2-pyrrolidone (NMP) and dimethyl sulfoxide (DMSO) at a weight:weight ratio of about 1:3 to about 3:1 (N-methyl 2-pyrrolidone:dimethyl sulfoxide),
wherein the composition exhibits a skin permeation rate of the diclofenac or salt of diclofenac that is at least five times greater than the skin permeation rate of the diclofenac or salt of diclofenac in the same composition that does not comprise the graft copolymer.

14. The composition of claim 13, wherein the graft copolymer is present at about 2% to about 3%, the diclofenac or salt of diclofenac is present at about 4% to about 6% and the skin permeation enhancer is present at about 10-20% N-methyl 2-pyrrolidone (NMP) and 20-30% dimethyl sulfoxide (DMSO).

15. The composition of claim 13, wherein the graft copolymer is present at about 2.5% to about 4%, the diclofenac or salt of diclofenac is present at about 2% to about 3% and the skin permeation enhancer is present at about 5-10% N-methyl 2-pyrrolidone (NMP) and 5%-10% dimethyl sulfoxide (DMSO).

16. A method of transdermally administering one or more non-steroidal anti-inflammatory drugs to an animal for systemic or local therapeutic effect, the method comprising:
a) applying a composition to the skin of the animal, the composition comprising:
i. about 1% to about 10% of a film forming hydrophilic graft copolymer, the graft copolymer being a reaction product of:
a. a polystyrene macromonomer having an ethylenically unsaturated functional group; and
b. at least one hydrophilic acidic monomer having an ethylenically unsaturated functional group,
wherein the weight percent of the polystyrene macromonomer in the graft copolymer is between about 1 and about 20%, and the weight percent of the total hydrophilic monomer in the graft copolymer is between 80 and 99%, wherein at least about 10% of said total hydrophilic monomer is acidic, said graft copolymer when fully hydrated having an equilibrium water content of at least 90%,
the graft copolymer being present in the composition in an amount sufficient to cause the composition to form a water swollen but insoluble jelly like mass upon contact with a biological environment;
ii. about 0.1% to about 10% of one or more non-steroidal anti-inflammatory drugs (NSAID); and
iii. about 2% to about 50% of a skin permeation enhancer selected from the group consisting of 2-pyrollidone, N-methyl 2-pyrrolidone (NMP), N-octyl 2-pyrrolidone, N-dodecyl-2-pyrrolidone, N-(2-hydroxyethyl)-2-pyrrolidone, dimethyl sulfoxide (DMSO), decylmethyl sulfoxide, and mixtures thereof; and
b) allowing the composition to dry by evaporation of volatile excipients,
wherein the composition exhibits a skin permeation rate of the non-steroidal anti-inflammatory drug that is at least five times greater than the skin permeation rate of the non-steroidal anti-inflammatory drug in the same composition that does not comprise the graft copolymer.

17. The method of claim 16, wherein the composition comprises about 1% to about 10% of a graft copolymer comprising poly(N,N-dimethylacrylamide-co-acrylic acid-co-polystyrene ethyl methacrylate), about 0.1% to about 10% of diclofenac or a salt of diclofenac and about 2% to about 50% of a skin permeation enhancer comprising a mixture of N-methyl 2-pyrrolidone (NMP) and dimethyl sulfoxide (DMSO) at a weight:weight ratio of about 1:3 to about 3:1 (N-methyl 2-pyrrolidone:dimethyl sulfoxide).

18. A method of treating inflammation and pain comprising applying the composition of claim 1 to a patient's skin in need of such treatment.

19. The method of claim 18, wherein the method is for systemic treatment of inflammation and pain.

20. The method of claim 18, wherein the method is for local treatment of inflammation and pain.
